(19) United States
(12) Patent Application Publication
(10) Pub. No.: US 2026/0225988 A1
(43) Pub. Date: Aug. 6, 2026

(54) METHOD FOR PREPARING ALKYL HYDROXYPROPANOATE

(71) Applicant: ZF Solution Co. Ltd., Gyeonggi-do (KR)

(72) Inventors: JAE YUN ROH, Gyeonggi-do (KR); JEONG WOOK LEE, Gyeonggi-do (KR)

(21) Appl. No.: 19/083,632

(22) Filed: Mar. 19, 2025

(30) Foreign Application Priority Data
Feb. 4, 2025 (KR) 10-2025-0013528

(51) Int. Cl. C07C 67/42

(52) U.S. Cl. CPC C07C 67/03; C07D 305/12

(57) ABSTRACT

In a method for preparing alkyl hydroxypropanoate, beta-propiolactone is reacted with alkyl alcohol in the presence of a heterogeneous basic catalyst. The produced alkyl hydroxypropanoate may be easily separated from the catalyst, and the catalyst can be reused in the reaction. The heterogeneous basic catalyst may be CaO or polystyrene-(1,8-diazabicyclo[5.4.0]undec-7-ene) (PS-DBU).

### CROSS-REFERENCE TO RELATED APPLICATION AND CLAIM OF PRIORITY

This application claims the benefit under 35 USC § 119 of Korean Patent Application No. 10-2025-0013528, filed on Feb. 4, 2025, in the Korean Intellectual Property Office, the entire disclosure of which is incorporated herein by reference for all purposes.

### BACKGROUND

### 1. Technical Field

The present invention relates to a method for preparing alkyl hydroxypropanoate.

### 2. Background Art

Alkyl 3-hydroxypropanoate (AHP) is an amphiphilic compound containing both hydrophilic and hydrophobic groups and is utilized for various applications. In particular, ethyl 3-hydroxypropanoate (EHP) is an important compound widely used in industrial fields such as polymer materials (e.g., acrylic, polyacrylonitrile (PAN), carbon fiber), electrolytes, pharmaceuticals, pesticides, flavoring production, and biodegradable plastics.

In conventional AHP manufacturing methods, a basic catalyst such as NaOH has been used. However, challenges arise in the separation process due to the homogeneous nature of the catalyst, leading to increased costs and reduced production efficiency. In particular, since NaOH, a homogeneous catalyst, dissolves in EHP, the product, additional steps such as fractional distillation are required for final product separation and purification, thereby increasing production costs.

Meanwhile, biological methods for producing AHP have also been reported. For example, US20160348137A1 discloses a method for producing 3-hydroxypropionic acid esters by culturing Acetobacter lovaniensis in a medium containing at least 1 g of phosphate per liter. However, these biological production methods present challenges for commercial production due to low yield and high moisture content. Additionally, their economic feasibility is limited as they require complex separation and purification processes.

Accordingly, there is a need to develop a method for producing AHP in an economical and efficient manner using a catalyst that can be easily separated from the product and reused.

### SUMMARY

The present invention is directed to a method for preparing alkyl hydroxypropanoate with high yield.

1. A method for preparing alkyl hydroxypropanoate, including the reaction of beta-propiolactone with an alkyl alcohol in the presence of a heterogeneous basic catalyst.

2. In the method described in section 1, the catalyst is CaO or PS-DBU.

3. In the method described in section 1, the alkyl group contains 1 to 10 carbon atoms.

4. In the method described in section 1, the produced alkyl hydroxypropanoate is separated from the catalyst, which is subsequently reused in the reaction.

5. In the method described in section 1, the reaction is conducted in a reactor in which the catalyst is immobilized.

The present invention facilitates the separation of the product from the catalyst by utilizing a heterogeneous basic catalyst.

The present invention enables the economical and efficient production of alkyl 3-hydroxypropanoate by allowing catalyst reuse.

### BRIEF DESCRIPTION OF THE DRAWINGS

FIG. 1 is a graph presenting the gas chromatography results after the reaction with the CTF-1 catalyst.

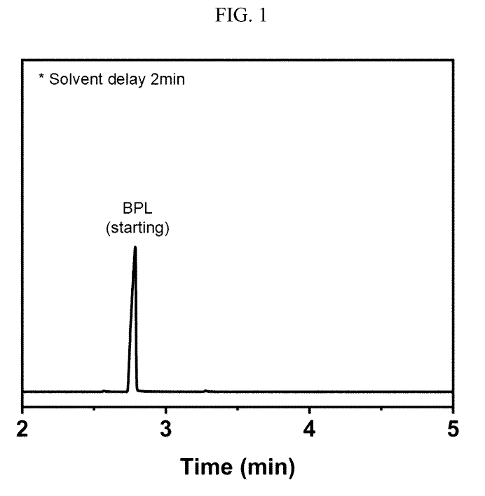

FIG. 2 is a graph presenting the gas chromatography results after the reaction with the TEA catalyst.

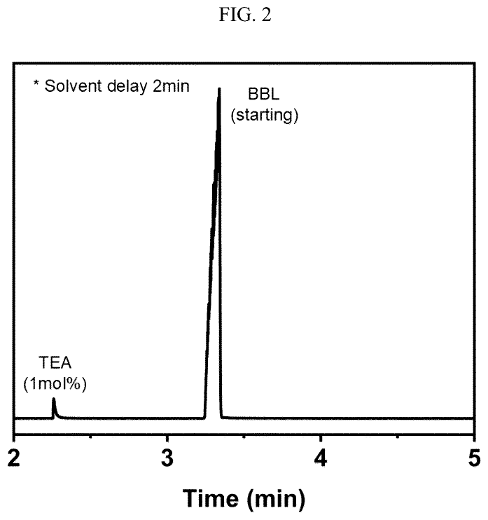

FIG. 3 presents the Nuclear magnetic resonance (NMR) and gas chromatography results after the reaction with the NaOH catalyst.

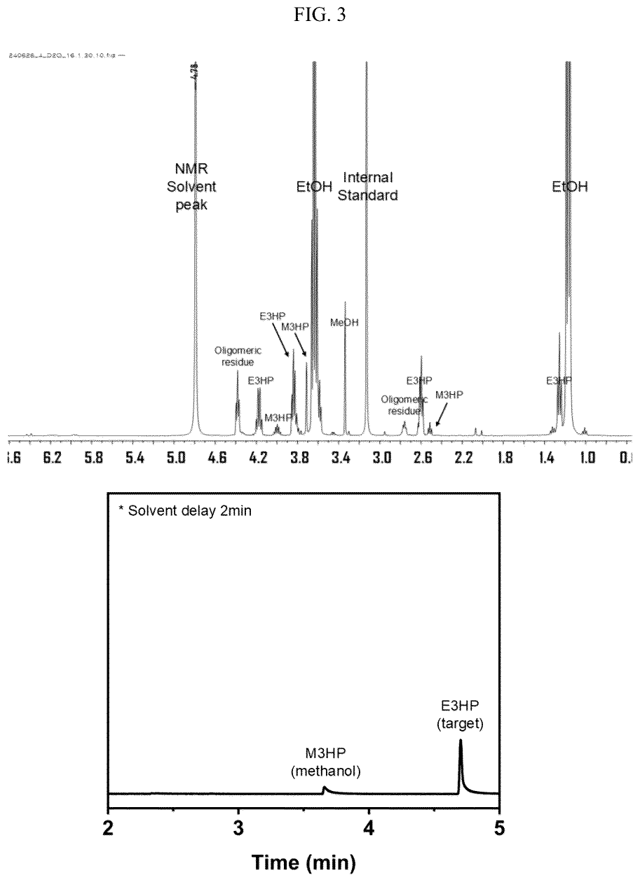

FIG. 4 presents the NMR results after the reaction with the Al(OH)₃ catalyst.

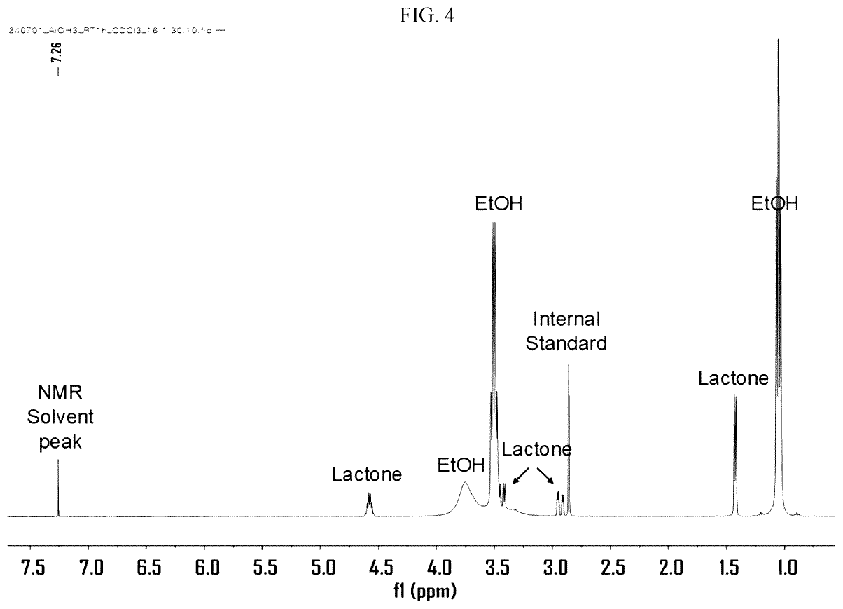

FIG. 5 presents the NMR results after the reaction in a benzene solution.

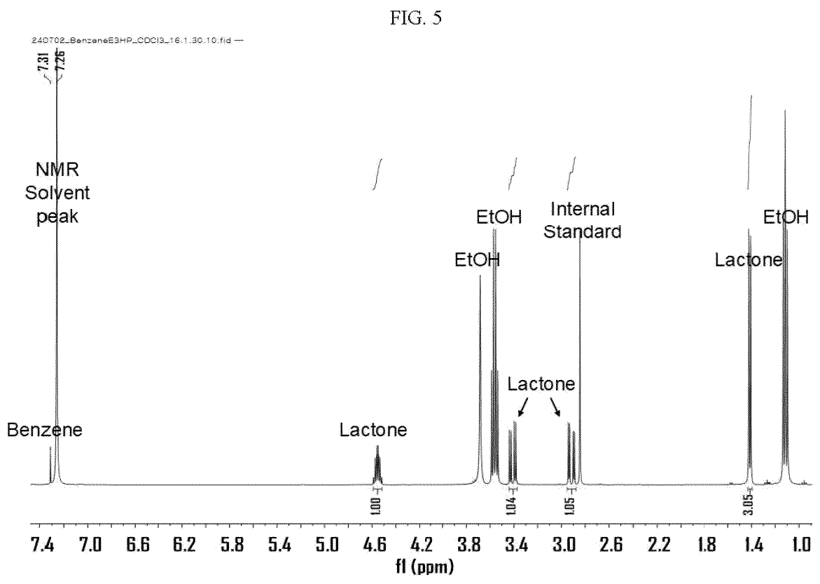

FIG. 6 presents the NMR results after the reaction with the 1,8-diazabicyclo[5.4.0]undec-7-ene (DBU) catalyst.

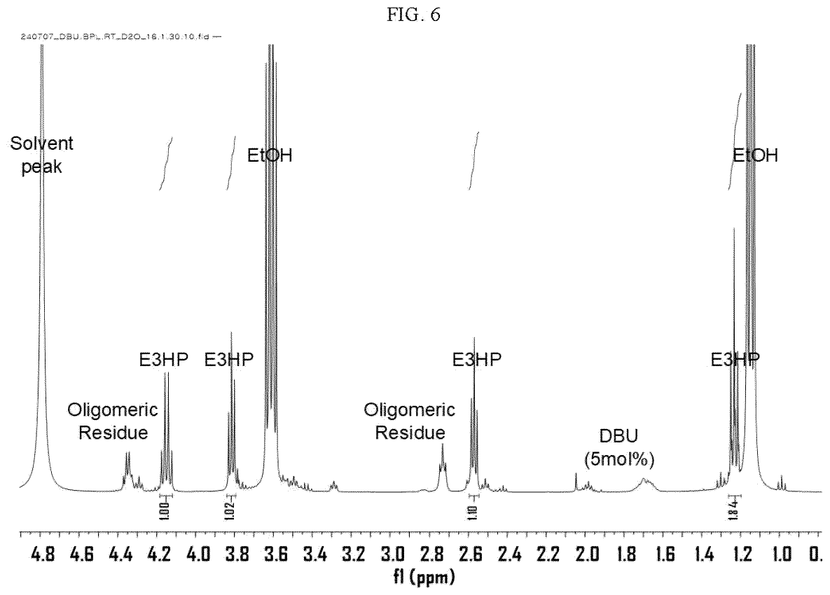

FIG. 7 presents the NMR results after the reaction with the CaO catalyst.

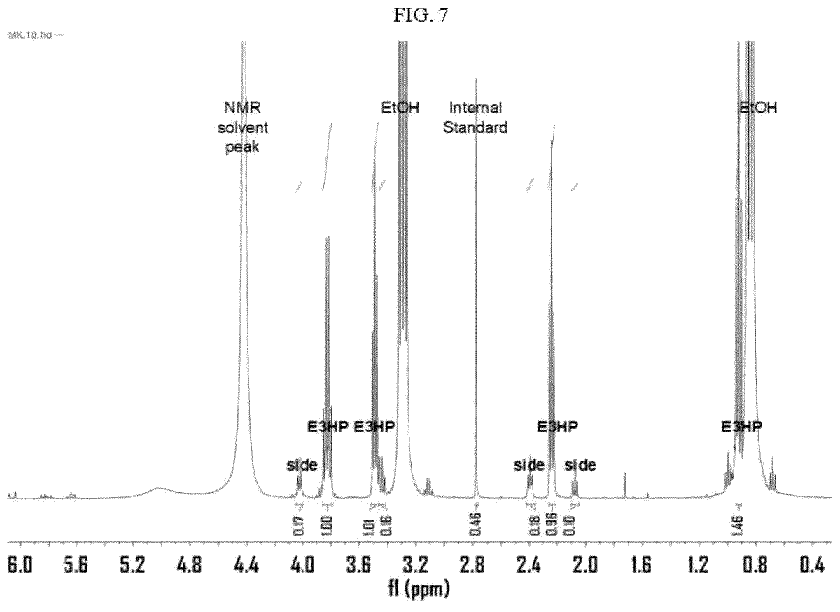

FIG. 8 presents the NMR results after the reaction with the polystyrene (PS)-DBU catalyst.

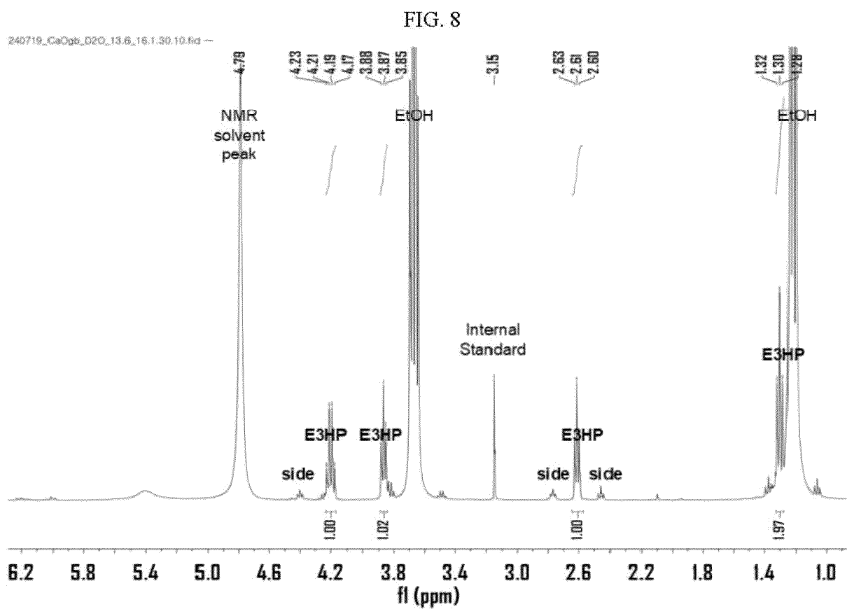

FIG. 9 presents the NMR results after the reaction with the PS-DBU catalyst reused after its first use.

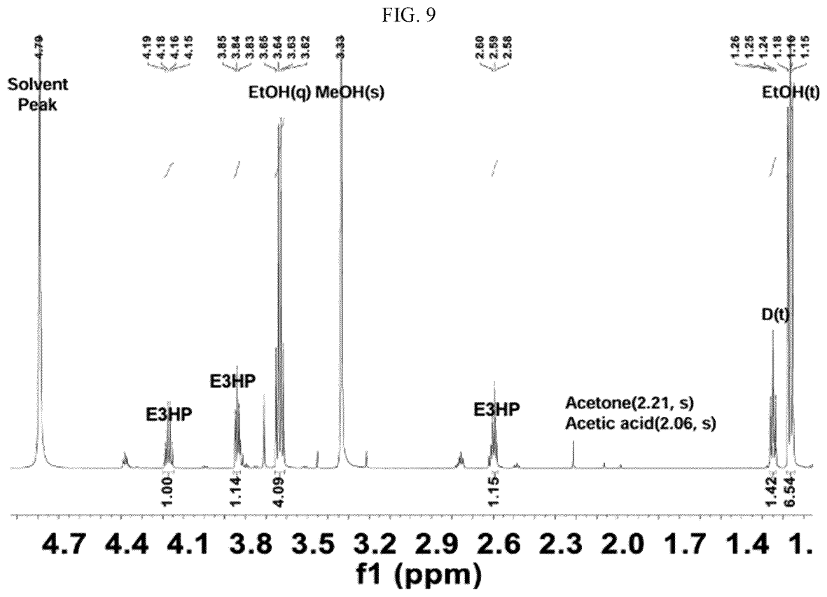

FIG. 10 presents the NMR results after the reaction with the PS-DBU catalyst reused after its second use.

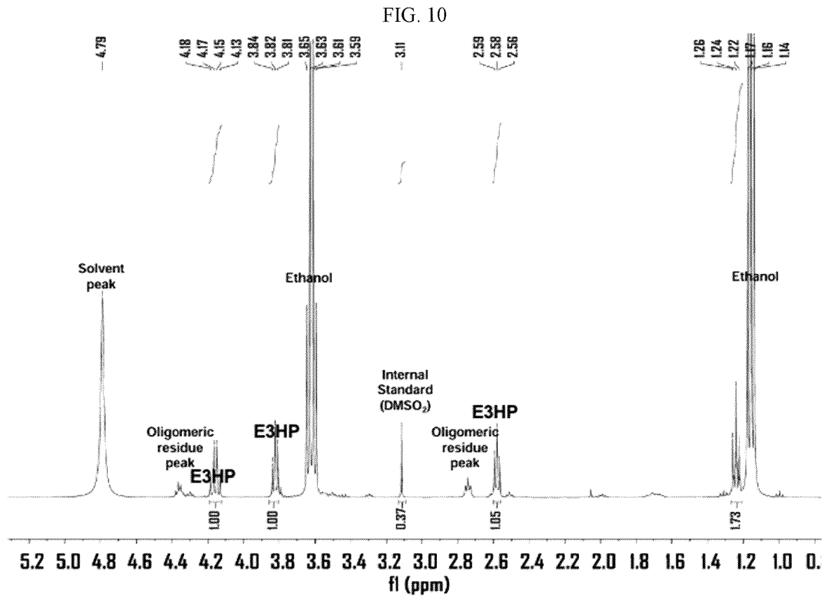

### DETAILED DESCRIPTION

The present invention is described in detail below.

The present invention relates to a method for producing alkyl hydroxypropanoate.

The method of the present invention includes the reaction of beta-propiolactone with an alkyl alcohol in the presence of a heterogeneous basic catalyst.

Conventionally, homogeneous catalysts such as NaOH have been used in the production of alkyl hydroxypropanoate. However, these catalysts dissolve in the product, making final product separation, purification, and catalyst reuse difficult. The present invention addresses these issues by employing a heterogeneous basic catalyst that remains insoluble in the product.

A heterogeneous basic catalyst is a solid-phase substance that exhibits basicity in reactions where the reactants and products are in the liquid phase.

These may include metal oxides, mixed metal oxides, or basic compounds immobilized on supports.

Examples of metal oxides include MgO, CaO, and ZnO. The metal oxides may be porous.

Examples of mixed metal oxides include Mg—Al mixed oxides and Ca—Zr mixed oxides.

Examples of suitable supports include polystyrene (PS), polymethyl methacrylate (PMMA), polyvinyl alcohol (PVA), polyvinylpyrrolidone (PVP), polytetrafluoroethylene (PTFE), polyarylene ether sulfone (PAES), silica, alumina, activated carbon, and zeolite.

The types and methods of bonding between the support and the basic compound are not specifically limited and may be based on known techniques. Examples of such bonding include covalent bonding, ionic bonding, coordination bonding, hydrogen bonding, physical adsorption, immobilization via covalent attachment, surface modification immobilization, polymer matrix entrapment, and electrostatic interaction-based immobilization. Bonding may also be facilitated through the use of crosslinkers. The basic compound may include, for example, DBU (1,8-Diazabicyclo[5.4.0]undec-7-ene), DBN (1,5-Diazabicyclo[4.3.0]non-5-ene), pyridine, alkylamines, NaOH, and KOH. Preferably, the catalyst may be MgO, CaO, DBU, or other similar basic compounds.

The reaction takes place between beta-propiolactone and an alkyl alcohol, wherein the alkyl alcohol may contain an alkyl group with 1 to 10, 1 to 5, or 1 to 3 carbon atoms.

The molar ratio of beta-propiolactone to alkyl alcohol is not particularly limited and is typically in the range of 1:1 to 1:10. The alcohol serves as both a reactant and a solvent, and when present in excess, it shifts the equilibrium toward product formation, thereby enhancing conversion. Accordingly, it is preferably used at a molar ratio of 1:3 to 1:8.

The catalyst may be used at a molar ratio of 0.01 to 0.2, 0.01 to 0.1, 0.01 to 0.08, or 0.03 to 0.08 relative to beta-propiolactone. If the catalyst amount is too low, the reaction rate decreases, whereas an excessive amount may compromise economic efficiency.

The reaction conditions are not specifically limited and may be adjusted based on process requirements.

The reaction may be conducted at a temperature ranging from −10° C. to 50° C. Excessively low temperatures slow the reaction rate, while excessively high temperatures may induce side reactions. Therefore, it is preferably carried out at a temperature ranging from 0° C. to room temperature.

The reaction time may range from 10 minutes to 24 hours, preferably from 30 minutes to 1 hour. If the reaction time is too short, achieving sufficient conversion becomes difficult, while an excessively long reaction time may reduce productivity.

The reaction may be conducted under stirring, with the stirring speed typically ranging from 200 to 800 rpm.

In the method of the present invention, the produced alkyl hydroxypropanoate may be separated from the catalyst, which can then be reused in the reaction.

The method of the present invention employs a heterogeneous basic catalyst that remains insoluble in the product. Accordingly, the alkyl hydroxypropanoate and the catalyst may be separated by methods such as filtration, and the recovered catalyst can be reused in the reaction.

Alternatively, in the method of the present invention, the reaction may be conducted in a reactor in which the catalyst is immobilized.

Alkyl hydroxypropanoate can be continuously produced by loading the catalyst into a fixed-bed reactor and continuously supplying beta-propiolactone and alkyl alcohol. This continuous process offers the advantages of eliminating separation steps and being well-suited for large-scale production.

According to the present invention, the use of a heterogeneous basic catalyst facilitates the separation of the product from the catalyst, enabling the economical and efficient production of alkyl 3-hydroxypropanoate through catalyst reuse. In particular, when using a PS-DBU catalyst, a conversion rate of over 99% can be maintained even after multiple reuses. Furthermore, continuous production in a reactor with an immobilized catalyst offers advantages for large-scale manufacturing. As a result, alkyl 3-hydroxypropanoate, essential in various industrial fields such as polymer materials, electrolytes, pharmaceuticals, agrochemicals, fragrances, and biodegradable plastics, can be efficiently supplied.

The present invention is further illustrated by the following examples.

### Examples

### Synthesis of Ethyl 3-hydroxypropanoate (EHP) from Beta-propiolactone (BPL) and Ethanol

Examples 1 to 5 demonstrate the reaction of BPL with ethanol using various catalysts. Except for Example 3, which used NaOH, the reaction proceeded minimally with the other catalysts. In the case of DBU, CaO, and PS-DBU, as presented in this invention, successful conversion to EHP was confirmed with a conversion rate of over 99%. Additionally, when using PS-DBU immobilized on polystyrene as a heterogeneous solid catalyst (Examples 8-10), high conversion rates were maintained even after reuse through simple filtration of the solid catalyst. This result enhances the potential for catalyst recovery and repeated reuse, which are key components of the manufacturing process. Therefore, this invention is expected to play a major role in the economical production of alkyl 3-hydroxypropanoate, particularly MHP and EHP, through the reaction of BPL and alcohol.

TABLE 1

| | | | Conversion | |
|---|---|---|---|---|
| Example | Catalyst | Solvent | rate(%) | Etc. |
| 1 | *CTF-1 | | <1 | |
| 2 | **TEA | | <1 | |
| 3 | NaOH | <5% | 99> | Oligomeric |
| | | MeOH/EtOH | | residue |
| 4 | Al(OH)₃ | | <1 | |
| 5 | — | Benzene | <1 | |
| 6 | **DBU | | 99> | Oligomeric |
| | | | | residue |
| 7 | CaO | | 99> | Oligomeric |
| | | | | residue |
| 8 | ****PS-DBU | | 99> | Oligomeric |
| | | | | residue |
| 9 | First-reused | | 99> | Oligomeric |
| | PS-DBU | | | residue |
| 10 | Second-reused | | 99> | Oligomeric |
| | PS-DBU | | | residue |

*CTF-1: Covalent Triazine Framework-1
**TEA: Triethylamine
***DBU: 1,8-Diazabicyclo[5.4.0]undec-7-ene
****PS-DBU: PS-1,8-Diazabicyclo[5.4.0]undec-7-ene

PS-DBU was prepared by the following method.

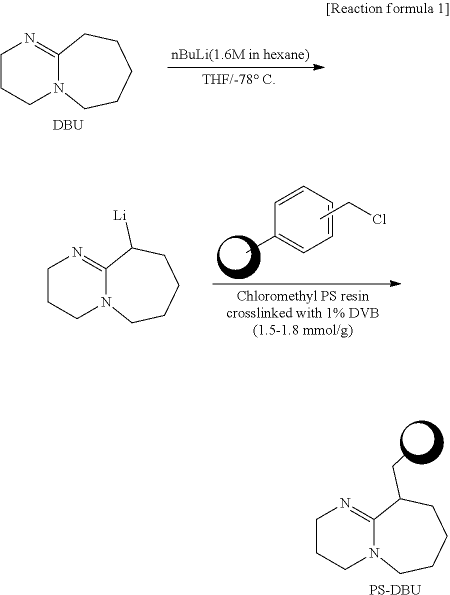

A 50 mL two-neck round-bottom flask was prepared, flame-dried, and purged with nitrogen three times. 7.5 mL of distilled THF and 0.76 mL of DBU were added using a syringe. Nitrogen purging was stopped, and an argon balloon was attached. A cooling bath at −78° C. was prepared using dry ice and acetone. 3 mL of n-BuLi was added very slowly to the round-bottom flask using a nitrogen-purged syringe (100→300 rpm). Subsequently, stirring was continued at 300 rpm for 2 hours. While rapidly purging with nitrogen again, chloromethyl PS (polystyrene) resin crosslinked with 1% DVB was quickly added. An argon balloon was reattached, and the reaction was carried out at 300 rpm for 36 hours while warming from −78° C. to room temperature. After the reaction, 10 mL of methanol was added to separate the polymer beads, which were washed sequentially with THF/methanol (1:1 volume ratio), methanol, water/methanol (1:1 volume ratio), acetone, and THE, then vacuum dried at 70° C.

Example 1 reacted BPL with ethanol in the presence of the CTF-1 catalyst. The molar ratio of reactants was BPL:ethanol:CTF-1=100:600:1, and the reaction was carried out for 1 hour at either an ice bath or room temperature, with stirring at 400 rpm. Gas chromatography analysis of the product showed that EHP was not produced.

Example 2 reacted BPL with ethanol in the presence of the TEA catalyst. The molar ratio of reactants was BPL:ethanol:TEA=100:600:1, and the reaction was carried out for 30 minutes at room temperature, with stirring at 400 rpm. Gas chromatography analysis of the product showed that EHP was not produced.

Example 3 reacted BPL with ethanol in the presence of the NaOH catalyst. The molar ratio of reactants was BPL:ethanol:NaOH=100:600:5, and the reaction was carried out for 30 minutes in an ice bath, with stirring at 400 rpm. NMR and gas chromatography analysis of the product showed that EHP was produced with over 99% conversion.

Example 4 reacted BPL with ethanol in the presence of the Al(OH)₃ catalyst. The molar ratio of reactants was BPL:ethanol:Al(OH)₃=100:600:5, and the reaction was carried out for 1 hour at room temperature, with stirring at 400 rpm. NMR analysis of the product showed that EHP was not produced.

Example 5 reacted BPL with ethanol in a benzene solution. The molar ratio of reactants was BPL:ethanol=1:2, and the volume ratio of ethanol to benzene was 1:1. The reaction was carried out for 24 hours at room temperature, with stirring at 400 rpm. NMR analysis of the product showed that EHP was not produced.

Example 6 reacted BPL with ethanol in the presence of the DBU catalyst. The molar ratio of reactants was BPL:ethanol:DBU=100:600:5, and the reaction was carried out for 30 minutes at room temperature, with stirring at 400 rpm. NMR analysis confirmed that EHP was obtained with a conversion exceeding 99%.

Example 7 reacted BPL with ethanol in the presence of the CaO catalyst. The molar ratio of reactants was BPL:ethanol:CaO=100:600:5, and the reaction was carried out for 30 minutes at room temperature, with stirring at 400 rpm. NMR analysis confirmed that EHP was obtained with a conversion exceeding 99%.

Example 8 reacted BPL with ethanol in the presence of the PS-DBU catalyst. The molar ratio of reactants was BPL:ethanol:PS-DBU=100:600:5, and the reaction was carried out for 30 minutes at room temperature, with stirring at 400 rpm. NMR analysis confirmed that EHP was obtained with a conversion exceeding 99%.

Example 9 reused the PS-DBU catalyst after its first use, which was recovered by filtration from Example 8, and used it to react BPL with ethanol. The molar ratio of reactants was BPL:ethanol:PS-DBU=100:600:5, and the reaction was carried out for 30 minutes at room temperature, with stirring at 400 rpm. NMR analysis confirmed that EHP was obtained with a conversion exceeding 99%.

Example 10 reused the PS-DBU catalyst after its second use, which had been recovered by filtration from Example 9, and subsequently used to catalyze the reaction of BPL with ethanol. The molar ratio of reactants was BPL:ethanol:PS-DBU=100:600:5, and the reaction was carried out for 30 minutes at room temperature, with stirring at 400 rpm. NMR analysis confirmed that EHP was obtained with a conversion exceeding 99%.

1: A method for preparing an alkyl hydroxypropanoate, the method comprising:
reacting beta-propiolactone with an alkyl alcohol in a presence of a heterogeneous basic catalyst at a temperature of −10° C. to 50° C. to produce the alkyl hydroxypropanoate,
wherein the alkyl alcohol participates in the reaction as a reactant and becomes incorporated into the alkyl hydroxypropanoate product, and
wherein the heterogeneous s basic catalyst is CaO or polystyrene-(1,8-diazabicyclo[5.4.0]undec-7-ene) (PS-DBU).

2. (canceled)

3: The method according to claim 1, wherein the alkyl has 1 to 10 carbon atoms.

4: The method according to claim 1, further comprising:
separating the produced alkyl hydroxypropanoate from the catalyst; and
recovering the catalyst and reusing the catalyst in a subsequent reaction cycle.

5: The method according to claim 1, wherein the reaction is performed in a reactor where the heterogeneous basic catalyst is fixed.

6: The method according to claim 1, wherein a molar ratio of the heterogeneous basic catalyst to the beta-propiolactone in the reaction is in a range of 0.03 to 0.08.
